(19) United States
(12) Patent Application Publication
(10) Pub. No.: US 2008/0004310 A1
(43) Pub. Date: Jan. 3, 2008

(54) LYOPHILIZED PHARMACEUTICAL COMPOSITION

(75) Inventors: Alton Samuel Kelley II, Mobile, AL (US); Jason R. Kelley, Mobile, AL (US); J. Michael Bennett, Mobile, AL (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: APPLIED PHARMACY SERVICES, INC., Mobile, AL (US)

(21) Appl. No.: 11/764,904

(22) Filed: Jun. 19, 2007

Related U.S. Application Data
(60) Provisional application No. 60/805,162, filed on Jun. 19, 2006.
(60) Provisional application No. 60/895,791, filed on Mar. 20, 2007.

(51) Int. Cl. A61K 31/472; A61K 31/4164; A61K 31/5575; A61P 15/10

(52) U.S. Cl. 514/307; 514/401; 514/573

(57) ABSTRACT

A stable pharmaceutical composition with increased solubility that includes at least one lyophilized compound that is papaverine, phentolamine or alprostadil.

### CROSS-REFERENCE TO RELATED APPLICATIONS

This application claims the benefit of U.S. Provisional Application Ser. No. 60/805,162 filed Jun. 19, 2006 entitled “Three Component Lyophilized Pharmaceutical Composition”, and U.S. Provisional Application Ser. No. 60/895,791 filed Mar. 20, 2007 entitled “Lyophilized Pharmaceutical Composition”, both of which are herein incorporated by reference.

### BACKGROUND

The present invention provides lyophilized pharmaceutical compounds and combinations of these compounds to treat erectile dysfunction and other vascular diseases, and methods for making the lyophilized compounds.

Erectile dysfunction may result from a variety of psychological and organic causes. Various methods for treating this condition are known. For example, U.S. Pat. Nos. 6,693,135, 6,890,945 and 7,041,677 provide a background of this condition and report a number of agents and methods of using these agents to treat erectile dysfunction. Each of these patents are incorporated herein by reference.

One method of treating erectile dysfunction uses an injectable solution of papaverine, phentolamine, and prostaglandin. Although this solution is effective, the active agents in the solution are unstable and degrade within a short period of time. The solutions of these agents that are currently used generally have a shelf life of about 60 days.

### SUMMARY

A stable pharmaceutical composition with increased solubility that includes at least one lyophilized compound that is papaverine, phentolamine, or alprostadil.

### DETAILED DESCRIPTION

Various embodiments of the present invention include pharmaceutical compositions, reconstituted injectable pharmaceutical solutions, methods of treating patients, and methods of making lyophilized compositions.

The present invention provides lyophilized compounds of papaverine, phentolamine, and prostaglandin, or combinations of these compounds that have an improved stability compared to a solution having the same active components. As used in this application, “composition” or “solution” refers to one or more lyphophilized compounds in various combinations according to alternative embodiments of this invention. The composition may include a single component of lyophilized papaverine, lyophilized phentolamine, or lyophilized prostaglandin; a combination of two components: lyophilized papaverine and lyophilized phentolamine, lyophilized papaverine and lyophilized phentolamine, or lyophilized phentolamine and lyophilized prostaglandin; or a combination of all three components: papaverine, phentolamine and prostaglandin. The active agents may be lyophilized in volumes in a range of about 0.5-10 ml.

An example of a particularly suitable prostaglandin includes, but is not limited to, alprostadil. In one embodiment, the pharmaceutical compositions of the present invention contain between about 1 μg/ml and about 120 μg/ml of alprostadil and preferably about 10 μg/ml of alprostadil. In another embodiment, the reconstituted solutions of the present invention contain between about 1 μg/ml and about 60 μg/ml of reconstituted alprostadil and preferably about reconstituted 15.2 μg/ml of alprostadil.

An example of a particularly suitable papaverine includes, but is not limited to, papaverine hydrochloride. In one embodiment, the pharmaceutical compositions of the present invention contain between about 0.1 mg/ml and about 90 mg/ml of papaverine hydrochloride and preferably about 30 mg/ml of papaverine hydrochloride. In another embodiment, the reconstituted solutions of the present invention contain between about 1 mg/ml and about 150 mg/ml of reconstituted papaverine hydrochloride and preferably about 60 mg/ml of reconstituted papaverine hydrochloride.

An example of a particularly suitable phentolamine includes, but is not limited to, phentolamine mesylate. In one embodiment, the pharmaceutical compositions of the present invention contain between about 0.01 mg/ml and about 10 mg/ml of phentolamine mesylate and preferably about 1 mg/ml of phentolamine mesylate. In another embodiment, the reconstituted solutions of the present invention contain between about 0.1 mg/ml and about 6 mg/ml of reconstituted phentolamine mesylate and preferably about 1.5 mg/ml of reconstituted phentolamine mesylate.

In addition to the three active agents, the pharmaceutical compositions of the present invention may also optionally include other known additives such as known pharmacologically acceptable excipients and solubilizing agents. Alternative embodiments of the present invention may also contain other ranges of the active agents to provide an amount that is effective to specifically provide the desired clinical benefit of alleviating the erectile dysfunction and inducing a therapeutic effect.

The lyophilized compounds are reconstituted with a “pharmaceutically acceptable solvent.” As used in this application, pharmaceutically acceptable solvents include water, physiological saline, phosphate-buffered saline or any other carriers known in the art. In one embodiment, about 3 ml of sterilized water may be added to a reconstituted lyophilized compound.

The method of making a lyophilized composition in an embodiment of the present invention involves a number of different temperature cycles that are performed under reduced pressures. Although this embodiment provides one example of suitable temperature cycles under reduced pressure, those of ordinary skill would readily understand that other alternative temperature cycles under reduced pressure would be suitable. In one embodiment, a solution including at least sterilized water and one lyophilized compound is added into a vial and the vial is topped with a stopper. The topped, but not sealed, vial is first pre-frozen or conditioned at about −40 degrees Celsius (° C.) for about 6 hours. After this initial step, the frozen material is subjected to a pressure of less than about 140 millimeters Mercury (mm Hg). The temperature is then raised in a series of controlled cycles. In a first 16 hour cycle, the temperature is raised to about −34° C. at a ramp rate that does not exceed about 1.5 degrees Celsius per minute (° C./minute). In a second 4 hour cycle, the temperature is raised to about −20° C. at a ramp rate the does not exceed about 0.4° C./minute. In a third 4 hour cycle, the temperature is raised to about −10° C. at a ramp rate that does not exceed about 1.4° C./minute. Finally, in a fourth 8 hour cycle, the temperature is raised to about 20° C. at a ramp rate that does no exceed about 0.7° C./minute. After these four cycles, the lyophilized material is sealed in the vial under vacuum and the pressure is returned to normal atmospheric pressure.

It has been found that by following the present lyophophilization method, the solubilities of the compounds unexpectedly increase when reconstituted. This is particularly true with regard to papaverine which generally has a solubility of about 30 mg/ml. After being lyophilized according to one embodiment of the present invention, the solubility of papaverine increased to about 90 mg/ml.

In one embodiment, a lyophilized composition may be used to treat a patient having erectile dysfunction. The lyophilized composition is formed by first providing an erectile inducing amount of at least one lyophilized compound selected from papverine hydrochloride, phentolamine mesylate, and alprostadil. The lyophilized composition is then reconstituted with a pharmaceutically acceptable solvent, such as sterilized water, to produce a reconstituted pharmaceutical solution. The reconstituted pharmaceutical solution is then administered to the patient. Once the lyophilized compounds are reconstituted, the reconstituted pharmaceutical solution may be administered to the patient by a variety of methods, including, but not limited to orally or by injection. In one embodiment, the composition is administered by injection. Any suitable method of injection, such as intracavernosal, intravenous, intraarterial, intramuscular, intraperitoneal, intraportal, intradermal or subcutaneous may be used.

In other embodiments of the invention, the compositions of the present invention are used for treating circulatory diseases, such as for treating ischemic heart disease. These compositions may be used as antihypertensive drugs, anticoagulative drugs, antiarrhythmic drugs or as peripheral vasodilators, including both cerebral vasodilator and renal vasodilator.

### EXAMPLES

The present invention is more particularly described in the following examples that are intended as illustrations only, since numerous modifications and variations within the scope of the present invention will be apparent to those skilled in the art. Unless otherwise noted, all parts, percentages, and ratios reported in the following examples are on a weight basis, and all reagents used in the examples were obtained, or are available, from the chemical suppliers described below, or may be synthesized by conventional techniques.

### Example 1

Sterile water (147.75 ml), papaverine hydrochloride powder (3 g), phentolamine mesylate powder (0.075 g), alprostadil solution (2.225 ml of a solution of 30 mg in 90 ml of sterile water) were mixed until a clear solution was obtained. Aliquots of the clear solution (3 ml) were filtered through a sterilizing 0.22 um filter into lyophilization vials (5 ml). The filled lyophilization vials were topped, but not sealed, with lyophilization stoppers and freeze-dried according the following procedure.

The topped vials were placed into a commercial freeze-drier. The temperature of the drier was lowered to about −40° C. and maintained at that temperature for at least 6 hours. The temperature of the drier was then raised to about −34° C. at a rate that did not exceed about 1.5° C./minute and a vacuum of less than about 140 mm Hg was applied with a vacuum pump for about 16 hours. After about 16 hours and while maintaining the vacuum at less than 140 mm Hg, the drier temperature was slowly raised to about −20° C. over about 4 hours at a rate that did not exceed about 0.4° C./minute. After the 4 hours, the drier temperature was again slowly raised to about −10° C. over about 4 hours at a rate that did not exceed about 1.4° C./minute. After the 4 hours, the temperature was again slowly raised to about 20° C. over about 8 hours at a rate that did not exceed about 0.7° C./minute. Following the three temperature increasing steps, the vials were sealed under vacuum, the freeze-drier was returned to normal atmospheric pressure, and the vials were removed from the freeze-drier.

### Example 2

In accelerated aging conditions, the active components of the lyophilized compositions of the present invention have an extended shelf live of as much as 120 days. The accelerated aging data for one, three, and four weeks is set out in Table 1. In Table 1, each week of accelerated aging corresponds to about one month of normal aging.

TABLE 1
Accelerated Aging Results

| | | | | |
|---|---|---|---|---|
| | | Expected | Assayed | % of |
| Analyte | Units | Conc. | Conc. | Expected |

Week One

| | | | | |
|---|---|---|---|---|
| Papaverine HCl | mg/ml | 30 | 19.7 | 65.7% |
| Phentolamine | mg/ml | 1.0 | 1.0 | 100% |
| Mesylate | | | | |
| Alprostadil | μg/ml | 10 | 10.5 | 105% |

Week Three

| | | | | |
|---|---|---|---|---|
| Papaverine HCl | mg/ml | 30 | 20.3 | 67.7% |
| Phentolamine | mg/ml | 1.0 | .92 | 92% |
| Mesylate | | | | |
| Alprostadil | μg/ml | 10 | 10 | 100% |

Week Four

| | | | | |
|---|---|---|---|---|
| Papaverine HCl | mg/ml | 30 | 23.4 | 78% |
| Phentolamine | mg/ml | 1.0 | 1.0 | 100% |
| Mesylate | | | | |
| Alprostadil | μg/ml | 10 | 9.5 | 95% |

Accelerated Conditions (40° C./75% RH)
The samples were stored at room temperature prior to reconstitution.

### Example 3

A 56 year old, 75 kilogram (kg) male patient is diagnosed with having erectile dysfunction. The patient is instructed to inject the lypholized preparation of 30 mg/ml papaverine, 1.0 mg/ml phentolamine mesylate, and 10 μg/ml alprostadil on a daily basis, if this is the intended frequency of sexual activity. The lyophilized papaverine, phentolamine, and alprostadil are prepared according to Example 1. The patient is examined after one month. He reports that he uses the drug prior to intercourse and that he maintains an erection for a satisfactory period after vaginal penetration in almost every attempt at intercourse. The patient considers the quality of his sexual relationship markedly improved.

### Example 4

A 55 year old, 70 kg male patient is apparently in good health. He presents complaints of a loss of sexual desire and frequent erectile dysfunction. He reports that although he is periodically interested in initiating sexual relations, on most occasions he is unable to maintain a high level of interest sufficiently long to initiate or to adequately respond to his wife's initiating sexual activity. He further indicates that he has difficulty maintaining an erection until completion of sexual intercourse. 1.0 mg/ml of lyophilized phentolamine is given to him. The reconstituted phentolamine is injected about 0.5 hours before the patient intended to engage in sexual activity. The patient is examined after one month. He reports that he takes the drug combination 2-3 times per week. After taking the drug composition he feels motivated to initiate sexual activity and has no difficulty maintaining an erection to completion of sexual intercourse.

### Example 5

Following the general method of treatment described in Example 3, non-critical variations are made to the composition of Example 3 such that only 10 μg/ml of lyophilized alprostadil is used. The same positive treatment effect is obtained.

### Example 6

Following the general method of treatment described in Example 3, non-critical variations are made to the composition of Example 3 such that only 30 mg/ml of lyophilized papaverine hydrochloride is used. The same positive treatment effect is obtained.

1. A stable pharmaceutical composition with increased solubility comprising at least one lyophilized compound selected from the group consisting of papaverine, phentolamine, and alprostadil.

2. The pharmaceutical composition of claim 1, wherein the composition comprises between about 0.1 mg/ml and about 90 mg/ml of papaverine hydrochloride.

3. The pharmaceutical composition of claim 1, wherein the composition comprises between about 0.01 mg/ml and about 10 mg/ml of phentolamine mesylate.

4. The pharmaceutical composition of claim 1, wherein the composition comprises between about 1 μg/ml and about 120 μg/ml of alprostadil.

5. The pharmaceutical composition of claim 1, wherein the composition comprises at least one lyophilized compound selected from the group consisting of about 30 mg/ml of reconstituted papaverine hydrochloride, about 1 mg/ml of reconstituted phentolamine mesylate, and about 10 μg/ml of reconstituted alprostadil.

6. The pharmaceutical composition of claim 1, wherein the composition is used to treat at least one of erectile dysfunction and circulatory disease.

7. An injectable pharmaceutical solution comprising an erectile inducing amount of at least one reconstituted lyophilized compound, wherein the lyophilized compound is selected from the group consisting of papaverine hydrochloride, phentolamine mesylate, and alprostadil.

8. The injectable pharmaceutical solution of claim 7, wherein the solution comprises between about 1 mg/ml and about 150 mg/ml of reconstituted papaverine hydrochloride.

9. The injectable pharmaceutical solution of claim 7, wherein the solution comprises between about 0.1 mg/ml and about 6 mg/ml of reconstituted phentolamine mesylate.

10. The injectable pharmaceutical solution of claim 7, wherein the solution comprises between about 1 μg/ml and about 60 μg/ml of reconstituted alprostadil.

11. The injectable pharmaceutical solution of claim 7, wherein the solution comprises at least one lyophilized compound selected from the group consisting of about 60 mg/ml of reconstituted papaverine hydrochloride, about 1.5 mg/ml of reconstituted phentolamine mesylate, and about 15.2 μg/ml of reconstituted alprostadil.

12. A method of treating a patient, the method comprising:
(a) providing an erectile inducing amount of a lyophilized composition comprising at least one lyophilized compound, wherein the lyophilized compound is selected from the group consisting of papaverine hydrochloride, phentolamine mesylate, and alprostadil;
(b) reconstituting the lyophilized composition with a pharmaceutically acceptable solvent to provide a reconstituted pharmaceutical solution; and
(c) administering the reconstituted pharmaceutical solution to the patient.

13. The method of claim 12, wherein the lyophilized composition comprises between about 1 mg/ml and about 150 mg/ml of papaverine hydrochloride.

14. The method of claim 12, wherein the lyophilized composition comprises between about 0.01 mg/ml and about 10 mg/ml of phentolamine mesylate.

15. The method of claim 12, wherein the lyophilized composition comprises between about 1 μg/ml and about 60 μg/ml of alprostadil.

16. The method of claim 12, wherein the lyophilized composition comprises at least one lyophilized composition selected from the group consisting of about 60 mg/ml of reconstituted papaverine hydrochloride, about 1.5 mg/ml of reconstituted phentolamine mesylate, and about 15.2 μg/ml of reconstituted alprostadil.

17. The method of claim 12, wherein the lyophilized composition is used to treat a circulatory disease.

18. A pharmaceutical dosage to treat a patient with erectile dysfunction, the pharmaceutical dosage comprising:
(a) at least one lyophilized composition selected from the group consisting of about 60 mg/ml of reconstituted papaverine hydrochloride, about 1.5 mg/ml of reconstituted phentolamine mesylate, and about 15.2 μg/ml of reconstituted alprostadil; and
(b) about 3 ml of sterilized water.

19. A method of reconstituting a lyophilized pharmaceutical composition to treat erectile dysfunction, the method comprising:
(a) adding sterile water and at least one lyophilized compound to a lyophilization vial, wherein the lyophilized compound is selected from the group consisting of papaverine hydrochloride, phentolamine mesylate, and alprostadil;
(b) topping the lyophilization vial with a lyophilization stopper to create a topped, but not sealed, lyophilization vial;
(c) placing the topped lyophilization vial in a freeze-drier, wherein the freeze-drier has a temperature and a pressure;
(d) lowering the temperature of the freeze-drier to about −40° C. for at least about 6 hours;
(e) lowering the pressure of the freeze-drier to less than about 140 mm Hg;
(f) raising the temperature of the freeze-drier to about −34° C. at a rate not exceeding about 1.5° C./minute over a period of about 16 hours;
(g) raising the temperature of the freeze-drier to about −20° C. at a rate not exceeding about 0.4° C./minute over a period of about 4 hours;
(h) raising the temperature of the freeze-drier to about −10° C. at a rate not exceeding about 1.4° C./minute over a period of about 4 hours;
(i) raising the temperature of the freeze-drier to about 20° C. at a rate not exceeding about 0.7° C./minute over a period of about 8 hours;
(j) sealing the vial under vacuum in the freeze-drier; and
(k) raising the pressure of the freeze-drier to atmospheric pressure.

20. The method of claim 19, wherein the lyophilized compound is papaverine, and wherein a solubility of the papaverine increases when the lyophilized pharmaceutical composition is reconstituted.
